(19) United States
(12) Patent Application Publication
(10) Pub. No.: US 2021/0113282 A1
(43) Pub. Date: Apr. 22, 2021

(54) SURGICAL ROBOTIC SYSTEMS

(71) Applicant: Covidien LP, Mansfield, MA (US)

(72) Inventor: Jaimeen Kapadia, Cambridge, MA (US)

(21) Appl. No.: 17/050,599

(22) PCT Filed: May 7, 2019
(86) PCT No.: PCT/US2019/031013

§ 371 Date: Oct. 26, 2020

Related U.S. Application Data
(60) Provisional application No. 62/669,126, filed on May 9, 2018.

(51) Int. Cl. A61B 34/30; B25J 9/10; B25J 15/02; B25J 5/00

(52) U.S. Cl. CPC A61B 34/30; B25J 5/00; B25J 15/0213; B25J 9/102

(57) ABSTRACT

An instrument drive unit for use in a robotic surgical system includes a carriage configured to be coupled to a robotic arm, a plurality of drive shafts rotationally supported in the carriage and configured to interface with a corresponding driven member of an electromechanical surgical instrument, a plurality of drive gears fixed to a corresponding drive shaft, a plurality of motors, and a plurality of motor gears operably coupled to a corresponding motor. Each motor gear is configured to rotate a corresponding drive gear in response to an activation of a corresponding motor to actuate a function of the electromechanical surgical instrument.

### BACKGROUND

Surgical robotic systems have been used in minimally invasive medical procedures. Some surgical robotic systems included a console supporting a surgical robotic arm and a surgical instrument having at least one end effector (e.g., forceps or a grasping tool) mounted to the robotic arm. The robotic arm provided mechanical power to the surgical instrument for its operation and movement.

Manually-operated surgical instruments often included a handle assembly for actuating the functions of the surgical instrument. However, when using a robotic surgical system, no handle assembly was typically present to actuate the functions of the end effector. Accordingly, to use each unique surgical instrument with a robotic surgical system, an instrument drive unit was used to interface with the selected surgical instrument to drive operations of the surgical instrument.

The instrument drive unit was typically coupled to the robotic arm via a slide. The slide allowed the instrument drive unit and the attached surgical instrument to move along an axis of the slide, providing a means for adjusting the axial position of the end effector of the surgical instrument.

### SUMMARY

In accordance with an aspect of the present disclosure, an instrument drive unit for use in a robotic surgical system is provided and includes a carriage configured to be coupled to a robotic arm, a plurality of drive shafts rotationally supported in the carriage, a plurality of drive gears, a plurality of motors, and a plurality of motor gears. The drive shafts are configured for interfacing with a corresponding driven member of an electromechanical surgical instrument, the drive gears are fixed to a corresponding drive shaft, and the motor gears are operably coupled to a corresponding motor. Each motor gear is configured to rotate a corresponding drive gear in response to an activation of a respective motor to actuate a function of the electromechanical surgical instrument.

In aspects, the instrument drive unit may further include a plurality of ring gears. The ring gears may operably couple a corresponding motor gear with a corresponding drive gear.

In another aspect, the ring gears may be vertically stacked.

In further aspects, a first of the ring gears and a first of the drive gears may be operably coupled to one another and aligned along a first horizontal plane; and a second of the ring gears and a second of the drive gears may be operably coupled to one another and aligned along a second horizontal plane, vertically displaced from the first horizontal plane.

The ring gears may be independently rotatable relative to one another.

In aspects, a first of the ring gears may have gear teeth on an inner periphery and an outer periphery thereof. The gear teeth on the inner periphery may interface with a corresponding drive gear, and the gear teeth on the outer periphery may interface with a corresponding motor gear.

In another aspect, the instrument drive unit may further include an inner housing rotatably supported in a longitudinally-extending channel defined by the carriage. The drive shafts may be rotationally supported in the inner housing and may be circumferentially spaced from one another.

In further aspects, one of the motor gears may be operably coupled to the inner housing to rotate the inner housing about a longitudinal axis extending through the channel of the carriage.

The inner housing may define a longitudinally-extending channel that is coaxial with the channel of the carriage. The channel of the inner housing may be dimensioned for receipt of a shaft of an electromechanical surgical instrument.

In another aspect, the drive gears may be vertically and horizontally offset from one another.

In further aspects, the motor gears may be vertically and horizontally offset from one another.

The instrument drive unit may further include a plurality of motor shafts extending from a corresponding motor. The motor gears may be fixed to a corresponding motor shaft.

In aspects, each of the drive shafts may have a proximal end portion configured for interfacing with a corresponding driven member of the electromechanical surgical instrument.

In another aspect of the present disclosure, an instrument drive unit for use in a robotic surgical system is provided and includes a carriage configured to be coupled to a robotic arm, a plurality of drive shafts rotationally supported in the carriage, a plurality of drive gears, a plurality of motors, a plurality of motor gears, and a plurality of vertically stacked ring gears. The drive shafts are circumferentially spaced from one another and configured for interfacing with a corresponding driven member of an electromechanical surgical instrument. The drive gears are fixed to a corresponding drive shaft and are disposed at a discrete vertical location relative to one another. The motor gears are operably coupled to a corresponding motor and disposed at a discrete vertical location relative to one another. The ring gears are disposed in the carriage and operably couple a corresponding motor gear with a corresponding drive gear, such that each motor gear is configured to rotate a corresponding drive gear in response to an activation of a respective motor to actuate a function of the electromechanical surgical instrument.

In yet another aspect of the present disclosure, a surgical robotic system is provided and includes a robotic arm, an elongated slide coupled to the robotic arm, and an instrument drive. The instrument drive unit includes a carriage configured to be coupled to the slide, a plurality of drive shafts rotationally supported in the carriage, a plurality of drive gears, a plurality of motors, and a plurality of motor gears. The drive shafts are configured for interfacing with a corresponding driven member of an electromechanical surgical instrument, the drive gears are fixed to a corresponding drive shaft, and the motor gears are operably coupled to a corresponding motor. The motor gears are configured to rotate a corresponding drive gear in response to an activation of a respective motor to actuate a function of the electromechanical surgical instrument.

In aspects, the instrument drive unit may further include a plurality of vertically stacked ring gears. The ring gears may operably couple a corresponding motor gear with a corresponding drive gear.

In further aspects, the instrument drive unit may further include an inner housing rotatably supported in a longitudinally-extending channel defined by the carriage. The drive shafts may be rotationally supported in the inner housing and may be circumferentially spaced from one another.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a surgical robotic system including an instrument drive unit coupled to a slide in accordance with the present disclosure;

FIG. 2 is a perspective view of the instrument drive unit of the surgical robotic system of FIG. 1 and an electromechanical instrument shown separated from the instrument drive unit;

FIG. 3 is a perspective view of the instrument drive unit of FIG. 2 and the electromechanical instrument shown coupled to the instrument drive unit;

FIG. 4 is a side cross-section view, taken along lines 4-4 in FIG. 2, of the instrument drive unit with the electromechanical surgical instrument removed;

FIG. 5 is a rear view of the instrument drive unit, with a carriage of the instrument drive unit shown in phantom, illustrating internal components of the instrument drive unit;

FIG. 6 is a side cross-section view, taken along lines 6-6 in FIG. 5, of the instrument drive unit with the electromechanical surgical instrument removed; and

FIG. 7 is a longitudinal cross-section view, taken along lines 7-7 in FIG. 4, of the instrument drive unit with the electromechanical surgical instrument removed.

### DETAILED DESCRIPTION

Embodiments of the presently disclosed surgical robotic system and instrument drive units thereof are described in detail with reference to the drawings, in which like reference numerals designate identical or corresponding elements in each of the several views. As used herein, the term “distal” refers to that portion of the surgical robotic system or component thereof that is closest to the patient, while the term “proximal” refers to that portion of the surgical robotic system or component thereof further from the patient.

As will be described in detail below, provided is an instrument drive unit of a surgical robotic system configured to allow for a top-loading of a surgical instrument. The instrument drive unit has a plurality of drive shafts each configured to be coupled to a corresponding driven member of the surgical instrument for carrying out a discrete function of the surgical instrument. The drive shafts of the instrument drive unit are operably coupled to a discrete motor of the instrument drive unit via a discrete transmission assembly. The configuration of the transmission assemblies allows for a reduction in the overall height of the instrument drive unit (e.g., the instrument drive unit is more compact). For example, gears of the transmission assemblies are vertically and horizontally offset from the gears of the other transmission assemblies. The instrument drive unit may also include a rotatable inner housing that rotationally supports the drive shafts. The inner housing is configured to be rotated via one of the motors to enable rotation of the attached surgical instrument about its longitudinal axis. Other features and benefits of the disclosed instrument drive units are further detailed below.

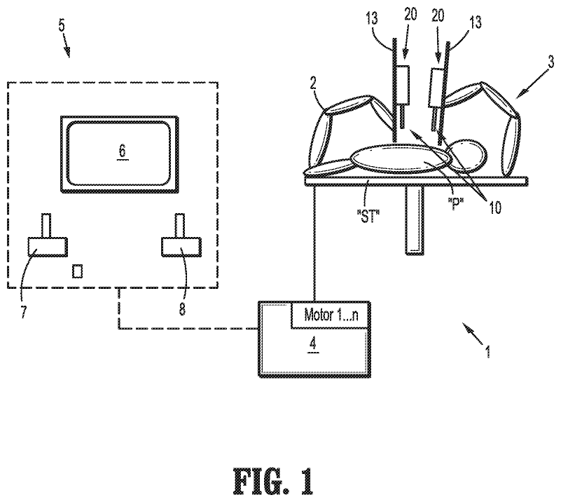

Referring initially to FIG. 1, a surgical system, such as, for example, a surgical robotic system 1, generally includes a plurality of surgical robotic arms 2, 3; an elongated slide 13 coupled to an end of each of the robotic arms 2, 3; an instrument drive unit 20 and an electromechanical instrument 10 removably attached to the slide 13 and configured to move along the slide 13; a control device 4; and an operating console 5 coupled with control device 4. The operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Each of the robotic arms 2, 3 may be composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, the attached instrument drive units 20, and thus electromechanical instrument 10 execute a desired movement according to a movement defined by means of manual input devices 7, 8. Control device 4 may also be set up in such a way that it regulates the movement of the instrument drive unit 20 along the slide 13, movement of the robotic arms 2, 3, and/or movement of the drives.

Surgical robotic system 1 is configured for use on a patient “P” lying on a surgical table “ST” to be treated in a minimally invasive manner by means of a surgical instrument, e.g., electromechanical instrument 10. Surgical robotic system 1 may also include more than two robotic arms 2, 3, the additional robotic arms likewise being connected to control device 4 and being telemanipulatable by means of operating console 5. A surgical instrument, for example, an electromechanical surgical instrument 10 (including an electromechanical end effector), may also be attached to the additional robotic arm.

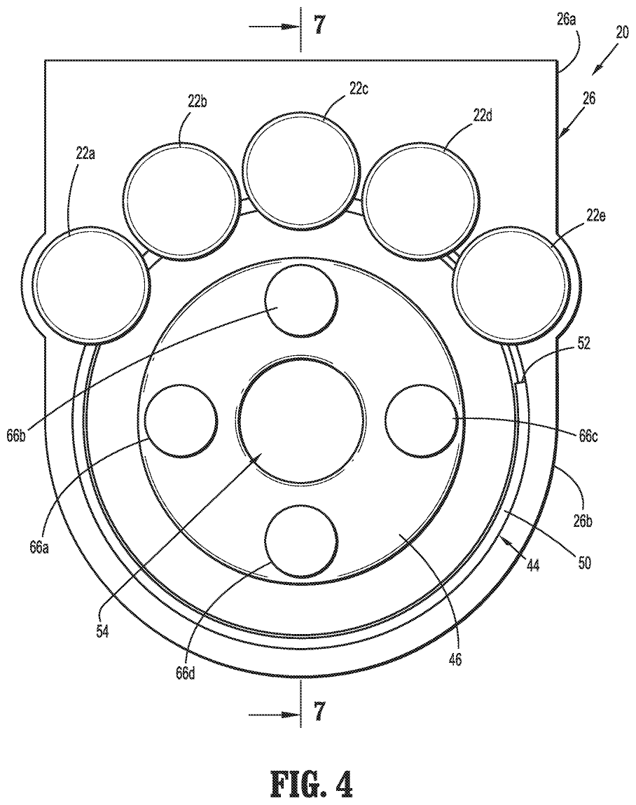

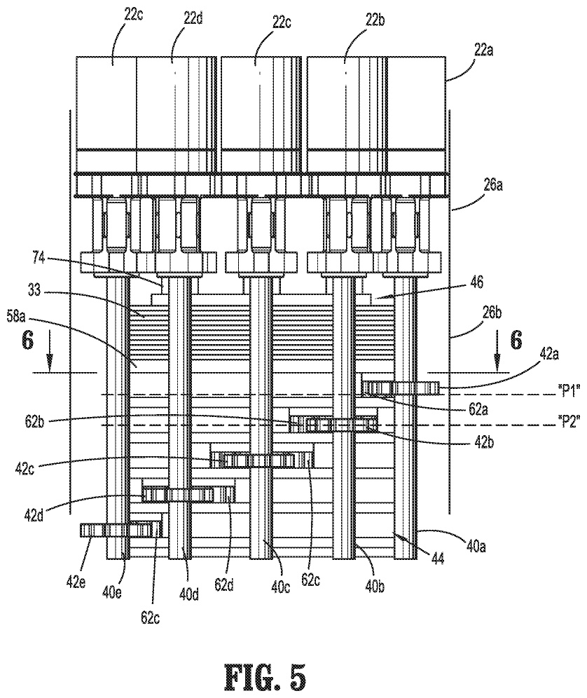

Control device 4 may control a plurality of motors, e.g., motors (Motor 1. . . n), with each motor configured to drive movement of robotic arms 2, 3 in a plurality of directions. Further, control device 4 may control a plurality of drive motors 22 (FIGS. 4 and 5) of the instrument drive unit 20 to drive various operations of the surgical instrument 10. The instrument drive unit 20 transfers power and actuation forces from its motors to driven members (not shown) of the electromechanical instrument 10 to ultimately drive movement of components of the end effector of the electromechanical instrument 10, for example, a movement of a knife blade (not shown) and/or a closing and opening of jaw members of the end effector.

For a detailed description of the construction and operation of a robotic surgical system, reference may be made to U.S. Pat. No. 8,828,023, entitled “Medical Workstation,” the entire contents of which are incorporated by reference herein.

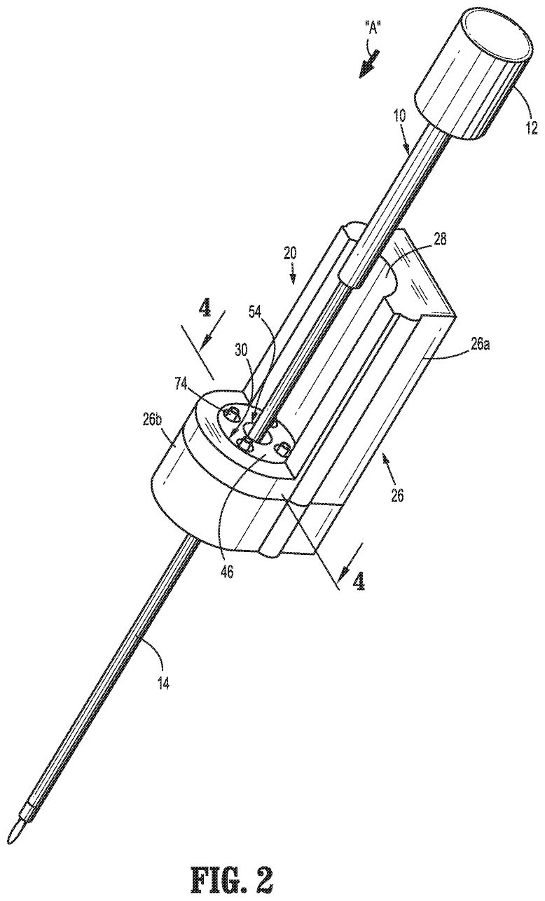

With reference to FIGS. 2-7, the instrument drive unit 20 will now be described in detail. The instrument drive unit 20 includes a carriage 26 having the plurality of drive motors 22a, 22b, 22c, 22d, 22d, 22e (collectively referred herein as “22”) disposed therein. The carriage 26 of the instrument drive unit 20 is configured to be slidably coupled to a linear track (not shown) defined longitudinally along the slide 13 (FIG. 1). The carriage 26 has a vertically-extending rear body 26a and a base 26b extending horizontally from a distal end of the rear body 26a. The rear body 26a of the carriage 26 defines an arcuate cutout 28 that extends along the length thereof and which is dimensioned to accommodate a slidable receipt of a main body portion 12 of the surgical instrument 10. The rear body 26a houses the plurality of motors 22 therein.

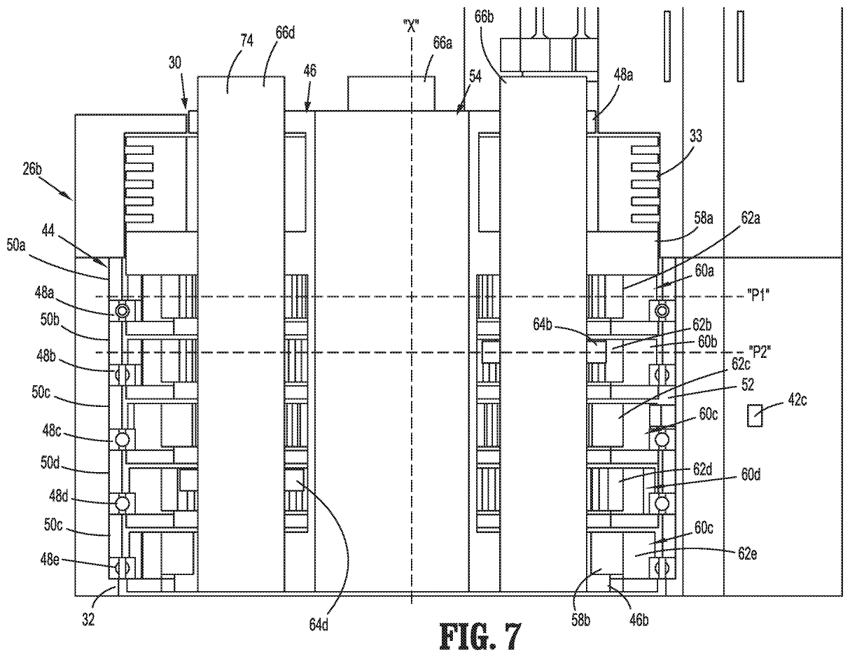

The base 26b of the carriage 26 defines a longitudinally-extending channel 30 therethrough dimensioned for receipt of an inner housing 46 of the instrument drive unit 20. The base 26b may have a semi-cylindrical shape. In embodiments, the base 26b may assume any suitable shape, such as, for example, squared. The base 26b has an annular ledge 32 (FIG. 7) that extends radially inward from an inner peripheral surface of the base 26b. The annular ledge 32 is configured to support internal components of the instrument drive unit 20.

In embodiments, a proximal end portion of the base 26b of the carriage 26 may have a slip ring 33 received therein for transferring electrical signals or power between fixed structures (e.g., the drive motors 22) and rotating structures (e.g., the electromechanical surgical instrument 10). The electrical signals transferred by the slip ring 33 may be feedback signals from the electromechanical surgical instrument 10 relating to the status and location of the surgical instrument 10 and/or the status and location of adjacent tissue structures. For example, the feedback may include the temperature of the surgical instrument 10, forces experienced by the surgical instrument 10, and/or the position of certain structures of the surgical instrument 10 relative to one another or relative to the adjacent tissue structures.

With reference to FIGS. 4 and 5, the drive motors 22 of the instrument drive unit 20 are concealed within the rear body 26a of the carriage 26. The drive motors 22 are circumferentially spaced from one another and are independently actuatable via the control device 4 (FIG. 1). One of the drive motors, such as, for example, drive motor 22e, is configured to effectuate a rotation of the surgical instrument 10 when the surgical instrument 10 is coupled to the instrument drive unit 20, and the remaining drive motors 22a, 22b, 22c, 22d are configured to actuate functions of the surgical instrument 10. While the instrument drive unit 20 is illustrated as having five drive motors, it is contemplated that the instrument drive unit 20 may have more or less than five drive motors.

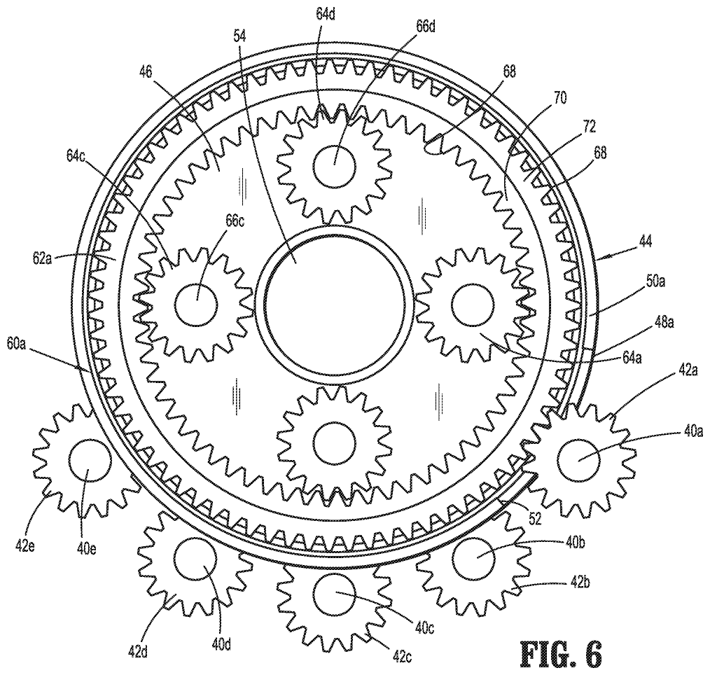

With reference to FIGS. 5 and 6, the drive motors 22 each have a rotatable motor shaft 40a, 40b, 40c, 40d, 40e (collectively referred to herein as “40”) extending distally therefrom and through the base 26b of the carriage 26. The motor shafts 40 are circumferentially spaced from one another about the channel 30 of the base 26b of the carriage 26. The motor shafts 40 each have a motor gear 42a, 42b, 42c, 42d, 42e (collectively referred to herein as “42”), such as, for example, a spur gear, rotationally fixed thereabout. Each of the motor gears 42 are positioned at a discrete vertical location on their respective motor shaft 40, such that the motor gears 42 are vertically offset a selected distance from one another. Since the motor gears 42, in addition to be vertically offset from one another, are also circumferentially spaced from one another, the motor gears 42 are offset from one another in all three dimensions.

With reference to FIGS. 4-7, the instrument drive unit 20 further includes an outer housing assembly 44 received in the channel 30 of the base 26b, and an inner housing 46 disposed within the outer housing assembly 44. The outer housing assembly 44 may be non-rotatably fixed to the base 26b of the carriage 26 and supported on the ledge 32 of the base 26b. As best shown in FIG. 7, the outer housing assembly 44 includes a plurality of bearings 48a, 48b, 48c, 48d, 48e (collectively referred to herein as “48”), or the like, and a plurality of ring supports 50a, 50b, 50c, 50d, 50e (collectively referred to herein as “50”) interposed between and fixed with adjacent bearings 48. The ring supports 50 and the bearings 48 are vertically stacked within the channel 30 of the carriage 26 in an alternating arrangement. A bottom-most (e.g., distal-most) bearing 48e sits on the ledge 32 of the base 26b to prevent the outer housing assembly 44 and, in turn, the inner housing 46, from falling distally through the channel 30. The ring supports 50 interconnect adjacent bearings 48, such that the entire outer housing assembly 44 is configured as a unitary structure. Each of the ring supports 50 has an opening 52 having a corresponding motor gear 42 extending therethrough to allow the motor gears 42 to interface with a corresponding ring gear 62, as will be described.

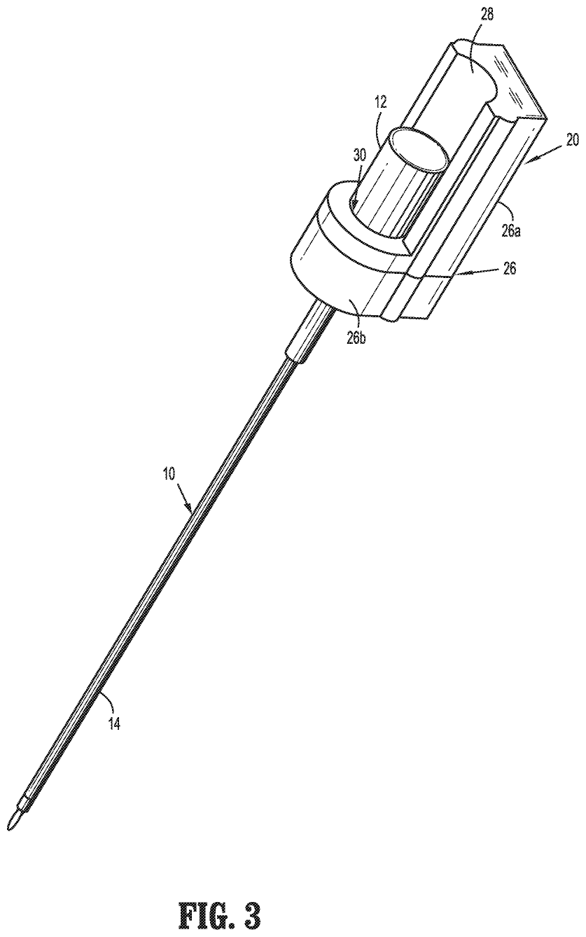

The inner housing 46 is supported in the outer housing assembly 44 and is configured to rotate relative to and within the outer housing assembly 44. The inner housing 46 defines an elongated lumen 54 that extends from a proximal end 46a to a distal end 46b thereof. The lumen 54 is dimensioned for slidable receipt of the shaft 14 (FIGS. 2 and 3) of the electromechanical surgical instrument 10. In some embodiments, the lumen 54 may be dimensioned to non-rotatably capture the shaft 14 of the electromechanical instrument 10 therein. The inner housing 46 has a pair of proximal and distal radial extensions 58a, 58b disposed adjacent respective proximal and distal ends 46a, 46b thereof. The radial extensions 58a, 58b axially support the inner housing 46 in the channel 30 of the base 26b of the carriage 26.

The instrument drive unit 20 further includes a plurality of transmission assemblies 60a, 60b, 60c, 60d, 60e (collectively referred to herein as “60”) that function independently from one another to transfer torque from a corresponding motor 22 to a corresponding driven member of the attached surgical instrument 10. Each transmission assembly 60a, 60b, 60c, 60d, 60e may include a respective motor gear 42, a ring gear 62a, 62b, 62c, 62d, 62e (collectively referred to herein as “62”), a drive gear 64a, 64b, 64c, 64d, 64e (collectively referred to herein as “64”), and a drive shaft 66a, 66b, 66c, 66d, 66e (collectively referred to herein as “66”) operably coupled to one another. In the embodiment shown, one of the transmission assemblies, such as, for example, transmission assembly 60e, is devoid of a drive gear because transmission assembly 60e functions to rotate the inner housing 46 of the instrument drive unit 20 rather than effectuate a function of the surgical instrument 10.

Components of the respective transmission assemblies 60 are vertically offset from one another along a longitudinal axis “X” defined through the lumen 54 of the inner housing 46, and certain components of each transmission assembly 60 are substantially aligned along a horizontal plane. For example, as best shown in FIGS. 5 and 7, the first motor gear 42a, the first ring gear 62a, and the first drive gear 64a of the first transmission assembly 60a (e.g., the proximal-most transmission assembly) are operably coupled to one another and substantially aligned along a first horizontal plane “P1,” and the second motor gear 42b, the second ring gear 62b, and the second drive gear 64b of the second transmission assembly 60b are operably coupled to one another and substantially aligned along a second horizontal plane “P2,” which is vertically displaced (e.g., disposed distally) from the first horizontal plane “P1” along the longitudinal axis “X.” The remaining transmission assemblies 60c, 60d, and 60e are also disposed in a discrete horizontal plane. While only five transmission assemblies are shown, it is contemplated that the instrument drive unit 20 may have more or less than five transmission assemblies.

The ring gears 62 of the transmission assemblies 60 are vertically stacked within the channel 30 of the carriage 26. In particular, the rings gears 62 are coaxial along the longitudinal axis “X” defined by the lumen 54 of the inner housing 46. The ring gears 62 are rotationally supported by a respective bearing 48 of the outer housing assembly 44. A distal-most (e.g., bottom-most) ring gear 62e is fixed to the distal radial extension 58b of the inner housing 46, such that a rotation of the distal-most ring gear 62e causes the inner housing 46 to rotate therewith and relative to the outer housing assembly 44.

Each of the ring gears 62 has gear teeth 68 extending from both an inner periphery 70 thereof and an outer periphery 72 thereof. The gear teeth 68 on the outer periphery 72 of each of the ring gears 62 interfaces with a corresponding motor gear 42, and the gear teeth 68 on the inner periphery 70 of each of the ring gears 62 interfaces with a corresponding drive gear 64, as will be described. In embodiments, each of the rings gears 62 may be constructed from inner and outer ring gears integrally formed with one another.

The drive shafts 66a, 66b, 66c, 66d of the transmission assemblies 60a, 60b, 60c, 60d extend longitudinally through the inner housing 46 and are circumferentially spaced from one another about the lumen 54 of the inner housing 46. The drive shafts 66 are free to rotate about their respective longitudinal axes in relation to the inner housing 46. The drive shafts 66 each have a proximal end portion 74 configured to operably couple to a driven member (not explicitly shown) of the surgical instrument 10. For example, the proximal end portion 74 of each of the drive shafts 66 may have a coupler (e.g., a gear) for coupling with a corresponding coupler of a driven member of the surgical instrument 10. Accordingly, upon top-loading of the electromechanical instrument 10 into the instrument drive unit 20, the proximal end portions 74 of each of the drive shafts 66 of the instrument drive unit 20 operably couple to the gears/couplers in a distal end of the main body portion 12 of the electromechanical instrument 10, such that a rotation of each drive shaft 66 rotates a correspondingly coupled driven member of the surgical instrument 10 to effectuate a discrete function of the surgical instrument (e.g., opening/closing of the end effector, articulation of the end effector, etc.)

The drive shafts 66 each have a drive gear 64 such as, for example, a spur gear, rotationally fixed thereabout. Each of the drive gears 64 are positioned at a discrete vertical location on their respective drive shaft 66, such that the drive gears 64 are vertically offset a selected distance from one another. Since the drive gears 64, in addition to being vertically offset, are also circumferentially spaced from one another, the drive gears 64 are offset from one another in all three dimensions. As mentioned above, the drive gears 64 each interface or intermesh with the gear teeth 68 on the inner periphery 70 of a corresponding ring gear 62 and receive torque therefrom originating from the respective motor 22.

In operation, as shown in FIGS. 2 and 3, the electromechanical instrument 10 is coupled to the instrument drive unit 20 by passing the shaft 12 of the electromechanical instrument 10 through the lumen 54 of the inner housing 46 of the instrument drive unit 20 in a distal direction, indicated by arrow “A” in FIG. 2. With the main body portion 12 of the electromechanical instrument 10 supported on the instrument drive unit 10, as shown in FIG. 3, the proximal end 74 of each of the drive shafts 66 of the transmission assemblies 60 interface with corresponding gears/couplers (not shown) in the distal end of the main body portion 12 of the electromechanical instrument 10.

With reference to FIGS. 5-7, to actuate a particular function of the surgical instrument 10, such as, for example, an opening or closing of an end effector of the surgical instrument 10, one of the drive motors 22 of the instrument drive unit 20, such as the first drive motor 22a, is activated via the control device 4 (FIG. 1). An activation of the first drive motor 22a rotates the first motor shaft 40a. Rotation of the first motor shaft 40a actuates the first transmission assembly 60a to transfer torque from the first motor shaft 40a to a first driven member of the electromechanical instrument 10.

In particular, the first motor gear 42a of the first transmission assembly 60a rotates with the first motor shaft 40a, which, in turn, rotates the first ring gear 62a and the first drive gear 64a of the first transmission assembly 60a. Since the first drive gear 64a is rotationally fixed about the first drive shaft 66a, a rotation of the first drive gear 64a causes the first drive shaft 66a to rotate, thereby rotating the first driven member of the electromechanical instrument 10 to actuate an associated function of the surgical instrument 10. The drive motor 22e may be configured to resist rotation of the motor shaft 40e thereof during actuation of any of the transmission assemblies 60a, 60b, 60c, 60d so that actuation of one of the transmission assemblies 60a, 60b, 60c, 60d does not inadvertently result in a rotation of the hub 46.

To rotate the electromechanical instrument 10 about its longitudinal axis, the fifth motor 22e of the instrument drive unit 20 is activated by the control device 4 (FIG. 1). An activation of the fifth motor 22e causes the fifth motor shaft 40e and the fifth motor gear 42e to rotate. Due to the fifth motor gear 42e being operably coupled to the fifth ring gear 62e, rotation of the fifth motor gear 42e causes the fifth ring gear 62e to rotate. Given that the fifth ring gear 62e is fixed to the inner housing 46, the inner housing 46 rotates with the fifth ring gear 62e about the longitudinal axis “X” and relative to the outer housing assembly 44 and the carriage 26. With the electromechanical instrument 10 non-rotationally supported in the lumen 54 of the inner housing 46 and the drive shafts 66 of the instrument drive unit 20 coupled with the driven members of the electromechanical instrument 10, the electromechanical instrument 10 rotates with the inner housing 46 relative to the carriage 26 to change a rotational orientation of the electromechanical instrument 10.

As can be appreciated, the instrument drive unit 20 described above improves usability of the surgical robotic system 1, reduces a foot-print of the overall system 1, improves safety architecture, and reduces the time required to remove surgical instruments in case of an emergency.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. An instrument drive unit for use in a robotic surgical system, the instrument drive unit comprising:
a carriage configured to be coupled to a robotic arm;
a plurality of drive shafts rotationally supported in the carriage, the plurality of drive shafts configured for interfacing with a corresponding driven member of an electromechanical surgical instrument;
a plurality of drive gears, each drive gear of the plurality of drive gears fixed to a corresponding drive shaft of the plurality of drive shafts;
a plurality of motors; and
a plurality of motor gears, each motor gear of the plurality of motor gears operably coupled to a corresponding motor of the plurality of motors, wherein each motor gear of the plurality of motor gears is configured to rotate a corresponding drive gear of the plurality of drive gears in response to an activation of a respective motor of the plurality of motors to actuate a function of the electromechanical surgical instrument.

2. The instrument drive unit according to claim 1, further comprising a plurality of ring gears, each ring gear of the plurality of ring gears operably coupling a corresponding motor gear of the plurality of motor gears with a corresponding drive gear of the plurality of drive gears.

3. The instrument drive unit according to claim 2, wherein the plurality of ring gears are vertically stacked.

4. The instrument drive unit according to claim 3, wherein a first ring gear of the plurality of ring gears and a first drive gear of the plurality of drive gears are operably coupled to one another and aligned along a first horizontal plane, and wherein a second ring gear of the plurality of ring gears and a second drive gear of the plurality of drive gears are operably coupled to one another and aligned along a second horizontal plane, vertically displaced from the first horizontal plane.

5. The instrument drive unit according to claim 2, wherein the plurality of ring gears are independently rotatable relative to one another.

6. The instrument drive unit according to claim 2, wherein at least a first ring gear of the plurality of ring gears has gear teeth on an inner periphery thereof and an outer periphery thereof, wherein the gear teeth on the inner periphery of the first ring gear interface with a corresponding drive gear of the plurality of drive gears, and the gear teeth on the outer periphery of the first ring gear interface with a corresponding motor gear of the plurality of motor gears.

7. The instrument drive unit according to claim 1, further comprising an inner housing rotatably supported in a longitudinally-extending channel defined by the carriage, wherein the plurality of drive shafts are rotationally supported in the inner housing and are circumferentially spaced from one another.

8. The instrument drive unit according to claim 7, wherein at least one of the plurality of motor gears is operably coupled to the inner housing to rotate the inner housing about a longitudinal axis extending through the channel of the carriage.

9. The instrument drive unit according to claim 8, wherein the inner housing defines a longitudinally-extending channel that is coaxial with the channel of the carriage, the channel of the inner housing being dimensioned for receipt of a shaft of an electromechanical surgical instrument.

10. The instrument drive unit according to claim 1, wherein the plurality of drive gears are vertically and horizontally offset from one another.

11. The instrument drive unit according to claim 1, wherein the plurality of motor gears are vertically and horizontally offset from one another.

12. The instrument drive unit according to claim 1, further comprising a plurality of motor shafts, each motor shaft of the plurality of motor shafts extending from a corresponding motor of the plurality of motors, each motor gear of the plurality of motor gears being fixed to a corresponding motor shaft of the plurality of motor shafts.

13. The instrument drive unit according to claim 1, wherein each drive shaft of the plurality of drive shafts has a proximal end portion configured for interfacing with a corresponding driven member of the electromechanical surgical instrument.

14. An instrument drive unit for use in a robotic surgical system, the instrument drive unit comprising:
a carriage configured to be coupled to a robotic arm;
a plurality of drive shafts rotationally supported in the carriage and circumferentially spaced from one another, the plurality of drive shafts configured for interfacing with a corresponding driven member of an electromechanical surgical instrument;
a plurality of drive gears, each drive gear of the plurality of drive gears fixed to a corresponding drive shaft of the plurality of drive shafts, the plurality of drive gears being disposed at a discrete vertical location relative to one another;
a plurality of motors;
a plurality of motor gears, each motor gear of the plurality of motor gears operably coupled to a corresponding motor of the plurality of motors, the plurality of motor gears being disposed at a discrete vertical location relative to one another; and
a plurality of vertically stacked ring gears disposed in the carriage, each ring gear of the plurality of ring gears operably coupling a corresponding motor gear of the plurality of motor gears with a corresponding drive gear of the plurality of drive gears, such that each motor gear of the plurality of motor gears is configured to rotate a corresponding drive gear of the plurality of drive gears in response to an activation of a respective motor of the plurality of motors to actuate a function of the electromechanical surgical instrument.

15. The instrument drive unit according to claim 14, further comprising an inner housing rotatably supported in a longitudinally-extending channel defined in the carriage, wherein the plurality of drive shafts are rotationally supported in the inner housing.

16. The instrument drive unit according to claim 15, wherein at least one of the plurality of motor gears is operably coupled to the inner housing to rotate the inner housing about a longitudinal axis extending through the channel of the carriage.

17. The instrument drive unit according to claim 14, further comprising a plurality of motor shafts, each motor shaft extending from a corresponding motor of the plurality of motors, each motor gear of the plurality of motor gears being fixed to a corresponding motor shaft of the plurality of motor shafts.

18. A surgical robotic system, comprising:
a robotic arm;
an elongated slide coupled to the robotic arm; and
an instrument drive including:
a carriage configured to be coupled to the slide;
a plurality of drive shafts rotationally supported in the carriage, the plurality of drive shafts configured for interfacing with a corresponding driven member of an electromechanical surgical instrument;
a plurality of drive gears, each drive gear of the plurality of drive gears fixed to a corresponding drive shaft of the plurality of drive shafts
a plurality of motors; and
a plurality of motor gears, each motor gear of the plurality of motor gears operably coupled to a corresponding motor of the plurality of motors, wherein each motor gear of the plurality of motor gears is configured to rotate a corresponding drive gear of the plurality of drive gears in response to an activation of a respective motor of the plurality of motors to actuate a function of the electromechanical surgical instrument.

19. The surgical robotic system according to claim 18, wherein the instrument drive unit further includes a plurality of vertically stacked ring gears, each ring gear of the plurality of ring gears operably coupling a corresponding motor gear of the plurality of motor gears with a corresponding drive gear of the plurality of drive gears.

20. The surgical robotic system according to claim 18, wherein the instrument drive unit further includes an inner housing rotatably supported in a longitudinally-extending channel defined by the carriage, the plurality of drive shafts rotationally supported in the inner housing and being circumferentially spaced from one another.
